(19) United States
(12) Patent Application Publication
(10) Pub. No.: US 2021/0265021 A1
(43) Pub. Date: Aug. 26, 2021

(54) SYSTEM AND METHOD FOR SIMULATING A CHEMICAL OR BIOCHEMICAL PROCESS

(71) Applicant: YF1, Nancy (FR)

(72) Inventors: Roger-Marc NICOUD, Lay-Saint-Christophe (FR); Etienne BERGER, Nancy (FR)

(73) Assignee: YF1, Nancy (FR)

(21) Appl. No.: 17/256,780

(22) PCT Filed: Jun. 25, 2019
(86) PCT No.: PCT/EP2019/066770

§ 371 Date: Dec. 29, 2020

(30) Foreign Application Priority Data
Jul. 3, 2018 (EP) 18181500.2

(51) Int. Cl. G16C 20/10; G06F 17/13; G06F 30/20

(52) U.S. Cl. CPC G16C 20/10; G06F 30/20; G06F 17/13

(57) ABSTRACT

A system for computer simulation of a chemical process includes at least a first operation which is described by an explicit algebraic equation. The system includes a receiving module for receiving the first explicit algebraic equation, a processing module which is configured to convert said explicit algebraic equation into an algebraic/differential equation and a resolution module which is configured to solve the algebraic/differential equation.

### TECHNICAL FIELD

The present invention pertains to the field of modelling and simulation in process engineering. In particular, the present invention relates to a simulation system and a simulation method for the computer-implemented simulation of a chemical or biochemical process.

### TECHNICAL STATE OF THE ART

It is frequently necessary for manufacturing companies in the fine chemicals and biotechnology sectors to perform estimations or comparisons of production processes involving multiple operations and various plants and equipment based on a very limited amount of data and information. This is especially the case in the early stages of development. It should be noted that in these industries, when a production process is to be conceived, it frequently so happens that the chances of successfully placing the target molecule on the market are very low, for example, for reasons such as failed clinical trials. There exist therefore a large number of technical and economic estimations to be made in respect of numerous molecules which will only be produced in small quantities and for a limited period of time.

When manufacturers simply wish to obtain a rapid simulation of a chemical or biochemical process in the absence of knowing specific information or details of the internal state of the machinery and equipment engaged in implementing the process, managers and engineers make performance estimations of the various different operations by relying on empirical knowledge or indeed simply even intuition. The types of said specific information or details to be known include: the content of compound A being output from the reactor is 50% without this content level being related to a specific information item/detail about the internal conditions of the reactor, or indeed the rate of recovery of compound A in the separator is 95% without this content level being related to a specific information item or detail about the internal conditions of the separator. These pre-established information items or details make it possible to relate the output quantities (also referred to as final quantities in transient state; hereinafter the terms final quantities or output quantities shall be used interchangeably) to the input quantities (also referred to as initial quantities in transient state; hereinafter the terms initial quantities or input quantities shall be used interchangeably). This method does not make use of material balances or heat balances and therefore does not take into account the internal states of the equipment. It is therefore not capable of predicting dynamics. On the basis of the details known of the input quantities of the process, it is possible to progressively calculate step by step, for each operation, the output quantities. It is therefore possible to determine, by making use of a simple calculator, such as an Excel spreadsheet for example, the output quantities obtained at the end of the process.

This method presents the advantages of simplicity and speed of computation.

However, it also presents several drawbacks:
the number of possible scenarios that may be envisaged is limited to the operators' experimental knowledge, thus optimisations and fine comparisons between the processes are therefore not possible;
when a number of operations are occurring in series during the process, the simulation becomes particularly complex and imprecise. This complexity is accentuated in the event of a compound recycling loop during the process;
transient state operations are not taken into account. This method is limited to continuous systems or to discontinuous batch systems in which transient state operations are not taken into account. However, very often the quantities produced by fine chemicals or biotechnology industry manufacturers do not allow for use of continuous production (unlike petrochemical industries, for example).

When manufacturers wish to determine with greater precision the final quantities and performance details of a process, it is necessary to resort to proven methods of process engineering, based on detailed modelling approaches, such as Honeywell UNISIM®, Aspen HYSYS®. These models entail the need to know a very large number of physical-chemical parameters, in particular thermodynamic parameters, which make it possible to simulate the behaviour and performance of the system as a function of internal conditions. These models take into account the material and heat balances and determine the evolution of internal state variables within the equipment. Although this method is more precise, it is very rarely used by fine chemicals or biotechnology industry manufacturers because it requires taking into account a large number of internal state variables and the use of very advanced numerical methods that are far too complex for non-specialists. The amount of time and resources required to determine the physical-chemical parameters and to develop the simulation tool are considered to be too great given the possibility of the process subsequently being used for only very small quantities.

None of the approaches that exist to date are entirely satisfactory. They are either limited to particularly simplistic scenarios, or too complex and require the determining of an extremely large number of parameters. There exists therefore a need for a simulation system and a simulation method for the computer-implemented simulation of a chemical or biochemical process that makes it possible to simulate complex scenarios, including a plurality of operations, within a reasonable time period while also limiting the number of physical-chemical parameters required for carrying out the simulation.

The present invention falls within this framework.

### SUMMARY

The embodiments of the invention provide an industrial tool for fine chemicals process engineering. A functional technical characteristic feature of the embodiments lies in the simulation of processes with a view to the subsequent implementation thereof in production facilities for producing chemicals or biochemicals.

The embodiments provide the means for predicting, in a concrete manner the behaviour of chemical or biochemical processes in order to perform a certain number of estimations, in particular as to their industrial feasibility. The embodiments of the invention thus provide the means for guiding the development of chemical or biochemical processes with sufficient precision, and within reasonable costs and time frames, so as to make it possible to estimate the chances of success of the industrial implementation thereof, and indeed to do so even prior to the installing of the plant and the commissioning of this plant.

An implementation in a purely intellectual sense, of a simulation of biochemical or chemical processes is not possible. This is in particular the case of simulations for industrial needs. In practice, it is impossible to carry out the calculations necessary to arrive at sufficiently precise and significant results that allow industrial decisions to be taken such as, for example, the installation of chemical plants. In addition, the implementation in a purely intellectual sense of the simulation would take a prohibitive amount of time, in particular for establishing or setting the parameters of the model.

However, without a computer-assisted simulation, it is impossible to conduct testing in a predictable manner or to effectively make a choice from among a plurality of projects of proposed chemical or biochemical processes those which offer the best performance, and moreover to do so within a reasonable time frame for industrial purposes.

There exist no purely intellectual, mathematical or even theoretical method that can be used for predicting in a rapid and exhaustive manner the behaviour of a fine chemicals process.

An aim of the invention is to enable the fine chemicals and biotechnology industries to use relevant simulators by providing a tool that can be used by a person who is not an expert in simulation, which allows for a proposed production project to be taken into account despite there initially being virtually no known details of the relevant physical-chemical aspects.

This tool should have the ability to help the user identify the sensitive points or limiting factors of the process under evaluation in order to determine the specific information or details to be acquired or to be used in order to achieve a given objective, to refine these predictions progressively as newly known information and data become available. In addition, this tool will also have to be reliable, easy to use, and provide for precision in the effective estimation of the technical-economic performance of the process being studied which is appropriate to the problem posed and which could evolve according to the needs and the evolution of the planned industrial project.

To this end, according to a first aspect, the invention relates to a system according to claim 1.

The invention makes it possible to obtain an algebraic-differential equation, the solution of which converges to a starting explicit algebraic equation. Thus, it is possible to use techniques for solving algebraic-differential equations in order to obtain solutions for algebraic equations. It is thus then possible to use the two types of modelling in a same given simulation system and to benefit from the advantages linked to the two types of equations in accordance with the choices made by the user for one part or another of the general global model of the chemical or biochemical process.

The system according to the invention makes it possible to simulate operations represented by means of explicit algebraic equations or algebraic-differential equations. In order to solve a system combining the two types of equations, the system according to the invention generates an algebraic-differential equation that converges to the solution of the explicit algebraic equation in a manner so as to allow merging of the equations into a single system of algebraic-differential equations.

According to a second aspect, the invention relates to a simulation method according to claim 4.

According to a third aspect, the invention relates to a computer program product comprising instructions which, upon the program being executed by a computer, lead to the latter carrying out the steps of the method according to the second aspect of the invention.

According to a fourth aspect, the invention relates to a non-transitory information storage medium that is computer-readable and comprises instructions which, when they are executed by a computer, lead to the latter carrying out the steps of the method according to the second aspect of the invention.

### Definitions

The term “fine chemicals” or also “specialty chemicals” is understood in this document to refer to a branch of the chemical industry that synthesizes specific products, in low production volumes, but with high added value, and which responds to high technical constraints, for example related to purity.

The term “biotechnology” is understood to refer to production technologies for producing molecules by means of fermentation, culturing of cells, extraction from the natural environment.

The term “algebraic equation” is understood to refer to an equation having one or more unknown real variables.

The term “differential equation” is understood to refer to an equation having one or more unknown functions; it takes the form of a relation between these unknown functions and their successive derivatives.

The term “explicit equation” is understood to refer to an equation between different variables where one variable is expressed explicitly in terms of other variables.

The term “implicit equation” is understood to refer to an equation between variables where no variable is expressed in terms of other variables.

The term “internal state variable,” is understood to refer to a chemical variable which describes what is occurring within the interior of a given plant or equipment, for example, the temperature, the pressure, the concentration, etc.

The terms “input state variable” or “output state variable” are understood to refer to a chemical variable that describes respectively, what is being input into or output from, a given equipment unit, for example the temperature, the pressure, the concentration, etc.

The term “pseudo internal state variable” is understood to refer to an artificial variable that makes it possible to transform simple “explicit algebraic equations” connecting a vector of output state variables to a vector of input state variables in a mathematical formalism that is identical to that of predictive models using “algebraic-differential equations”.

The term “predictive model” is understood to refer to the combination of a certain number of algebraic and differential equations based on concepts of chemistry and/or physics, the solution of which serves to enable the determination of the internal state variables of at least one operation carried out in at least one plant or equipment.

The term “operation” is understood to refer to a chemical or biochemical transformation enabling the passing from an input state to an output state (or from an initial state to a final state). Generally speaking, a chemical or biochemical process comprises a plurality of operations.

The term “characteristic time” is understood to refer to the lead time for the operation to get underway which, for chemical processes, varies for example from a few minutes to a few hours.

### DESCRIPTION OF THE FIGURES

FIG. 1 illustrates a chemical process that effectively implements a plurality of operations O1, O2, O3, O4 and a plurality of equipment units E1, E2, E3, E4.

FIG. 2 schematically illustrates the system in accordance with one embodiment of the invention.

FIG. 3 is a diagram illustrating the steps for performing the simulation in accordance with one embodiment of the invention.

FIG. 4 presents a block diagram representing a device for implementing one or more embodiments of the invention.

FIG. 5 represents a reaction scheme comprising a reactor with one input In, and two outputs, Out 1, Out2.

### DETAILED DESCRIPTION

A chemical method may be divided into a plurality of operations in a plurality of equipment units. The number of operations may be less than, greater than or equal to the number of equipment units. Indeed, the number of operations may be greater than the number of equipment units if multiple operations are carried out in the same equipment unit while the number of equipment units may be greater than the number of operations if an operation requires a plurality of equipment units.

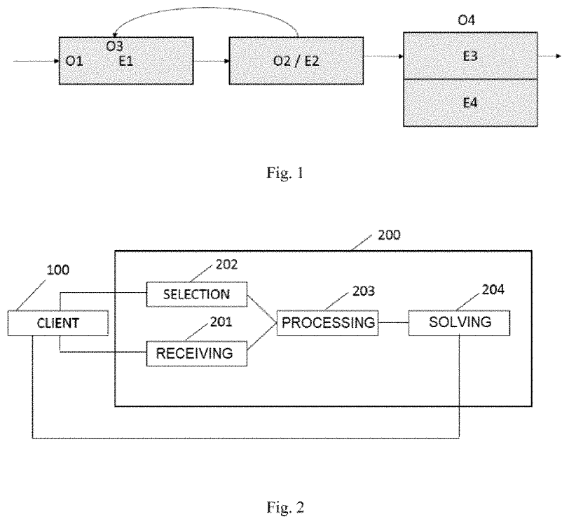

FIG. 1 illustrates these different case scenarios. On account of the feedback loop, the operations O1 and O3 are carried out within the same equipment unit E1 while the operation O4 is carried out with the aid of two equipment units E3 and E4 (by way of non-limiting example the equipment unit E3 may be a reactor and the equipment unit E4 may be a heat exchanger).

An operation (i) and an equipment unit (j) serve as means to pass over from an input state (Yₑ^{i/jj}) to an output state (Yₛ^{i/jj}). The input and output state variables are vectors representing the input and output chemical variables/values for an operation and an equipment unit of a chemical or biochemical process.

When a simple and rapid simulation of an operation and an equipment unit is desired, the operation and the equipment unit are described by means of explicit algebraic equations linking the input (Yₑ^{i/jj}) and output (Yₛ^{i/jj}) states for the operation (i) and the equipment unit (j): (Yₑ^{i/jj})=Gₑ (Yₑ^{i/jj}). There is no predictive ambition in this approach.

When a more complete and precise simulation is desired, it is necessary to determine what is happening within the interior of the equipment unit, and therefore to determine a vector of internal state variables (X^{i/j}) for an operation (i) and an equipment unit (j). The internal state variables are vectors representing internal chemical variable values for an operation and an equipment unit of a chemical or biochemical process.

In fine chemistry and/or biotechnology, the equipment units generally operate in a transient state, the internal state variables are therefore most often solutions of systems of algebraic and differential equations (algebraic-differential system) which represent material balances, heat balances or thermodynamic equilibria, for example.

In the general case, the operation and the equipment unit are described by a system of differential equations involving the internal state variables. These equations relate the vector of internal state variables (X^{i/j}) to the vector of input state variables (Yₑ^{i/jj}) for the operation (i) and the equipment unit (j):

$${P}^{E} \text{?} ( Y \text{?} , t , X \text{?} , \frac{d x \text{?}}{d T} ) = 0.$$

$$\text{?} \text{indicates text missing or illegible when filed}$$

The vector of output state variables (Yₛ^{i/jj}) which may depend on time is then determined explicitly based on the details of internal state variables being known:

Yₛ^{i/jj}(t)=P_{RT}^{E} (t, X^{i/j}) itself dependent on the vector of input state variables.

It should be noted that in steady state the final state of the vector of output state) variables is obtained immediately by: Y_{S}^{i/j}(t=∞)=P_{RT}^{E}(t=∞,X^{i/j})

where t_{∞} represents the end time of the operation in a discontinuous system or a sufficiently large time in a continuous system.

The equations describing the operations and equipment units of a chemical or biochemical process must be solved in a coupled manner by taking into account the connections between all the operations and all the equipment units during the entire process. In the case of a system of equations involving internal state variables, the solution thereof could be particularly long and time-intensive on account of issues related to convergence but above due to the need to know the relevant physical-chemical parameters the determination whereof could also be time-intensive and difficult.

Two modes of simulation for operations/equipment are thus available. One of these which is fairly trivial is based on simple explicit algebraic relationships between output and input state variables. The other, which is far more precise and predictive, requires the resolution of complex algebraic differential equations in addition to knowing the specific details of numerous physical-chemical parameters. It is therefore desirable to be able to move from a detailed approach that makes use of equations involving internal state variables to a simplified approach that uses explicit algebraic equations without the involvement of internal state variables for different operations within the same process. It would thus be possible to save precious time by choosing to simulate within a chemical or biochemical process, an operation that is simple or of secondary impact, by making use of an explicit algebraic equation, and an operation that is more complex or indeed critical, by making use of an algebraic-differential system involving internal state variables. However, due to the mathematical structure of the equations, it is not possible to integrate these two types of equations within the same general solving system.

In order to enable the integration of these two types of equations within a single solving system, the inventors developed the idea of replacing the explicit algebraic equations by algebraic-differential equations involving pseudo internal state variables. The algebraic-differential equations thus created are assigned a very low time constant compared to the characteristic time of the operation. It should be noted that a person skilled in the art would know the characteristic time of each operation of the method and would know how to choose a time constant that is low as compared to this characteristic time (for example a time constant of the order of a second). By selecting a low time constant, for a characteristic time that is far greater than this time constant, the differential term cancels out in steady state and it only remains to ensure that the other terms of the differential equation converge to the solution of the explicit algebraic equation.

Thanks to this transformation, the explicit algebraic equations may be integrated into the simulation system and the user is able to model for each operation, choosing between refined modelling or otherwise. Thus, while going from an algebraic system to an algebraic-differential system may seem to make the simulation more complex, it in fact serves to add a degree of flexibility to the simulation without penalizing computational performance. The user may opt for one or the other of the modelling systems, by using the same equation solving (resolution) module for solving equations.

By way of illustration, the explicit algebraic equation Yₛⁱ=Yₑ^{j} may be replaced by the differential equation

$$Θ \frac{d \tilde{{X}^{i}}}{d T} - α {Y}_{e}^{i} + {\tilde{X}}^{i} = 0 ,$$

with as initial condition \tilde{X}^{l} (t=0)=0, where Θ is a time constant and \tilde{X}^{l} is a pseudo internal state variable. When t is much greater than the time constant, the solution \tilde{X}^{l}(t) is constant, its derivative is therefore zero, and consequently αYₑⁱ=\tilde{X}ⁱ (t>>Θ). For a time sufficiently long as compared to the time constant, the solution of the explicit algebraic equation is thus found; in order to do this it suffices to set: Yₛⁱ=\tilde{X}ⁱ(t>>Θ).

The operator can therefore choose to describe an operation by using a system of equations involving the internal state variables or indeed an explicit algebraic equation. If at least one explicit algebraic equation is selected for at least one operation of the method, the system according to the invention replaces this explicit algebraic equation with an algebraic-differential equation in order to be able to integrate it into the system of equations for the entirety of the process.

FIG. 2 illustrates a block diagram of the system according to one embodiment of the invention. In the general context of the implementation of the system, a client device 100 connects to the simulation system 200 according to the invention. The system 200 according to the invention comprises a receiving module 201, optionally a selection module 202, a processing module 203, and an equation solving module (resolution module) for solving algebraic-differential equations 204.

The client device 100 connects to the receiving module 201 in order to communicate to the receiving module 201 an explicit algebraic equation that represents a chemical or biochemical operation and relates a vector of input state variables representing the initial chemical variable values of the said operation to a vector of output state variables representing the final chemical variable values of the said operation.

The receiving module 201 is connected to the processing module 203 in order to receive the explicit algebraic equation representing a chemical and biochemical operation and to create an algebraic-differential equation such that the steady-state solution of the algebraic-differential equation thus created converges to the said vector of output state variables according to the explicit algebraic equation.

The processing module 203 is connected to the equation solving module 204 for solving the algebraic-differential equation. When the processing module 203 has created the algebraic-differential equation, the equation solving module 204 solves the equation in order to obtain the vector of output state variables for the operation.

The algebraic-differential equation solving module 204 is connected to the client device 100 in order to return the vector of output state variables of the operation.

According to one embodiment, the client device 100 communicates to the receiving module 201 an explicit algebraic equation of the first operation and an algebraic-differential equation of the first operation. The client device 100 is connected to the selection module 202 so as to select a simulation using the explicit algebraic equation or a simulation using the algebraic-differential equation. Depending on the mode selected, the processing module creates an algebraic-differential equation converging to the said vector of output state variables according to the explicit algebraic equation or else uses the existing algebraic-differential equation.

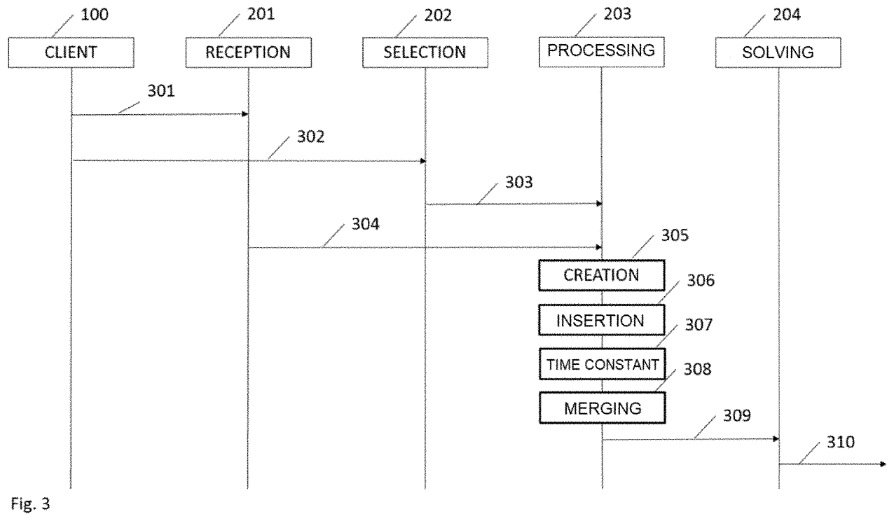

FIG. 3 illustrates the steps for performing the simulation in accordance with the embodiments.

The client module 100 communicates with the receiving module 201 in the step 301. During this step 301, the client module communicates to the receiving module an explicit algebraic equation that represents a first chemical or biochemical operation, and relates a first vector of input state variables representing the initial chemical variable values of the said first operation to a first vector of output state variables representing the final chemical variable values of the said first operation. According to one embodiment, during this step 301, the client device can also communicate an algebraic-differential equation representing the said first chemical or biochemical operation and relates a first vector of input state variables; and a first vector of internal state variables of the said first operation as an unknown of the equation. According to one embodiment, during this step 301, the client device can also communicate an algebraic-differential equation that represents a second chemical or biochemical operation and relates a second vector of input state variables; and a second vector of internal state variables of the said first operation as an unknown of the equation.

According to one embodiment, the client module 100 communicates with the receiving module in the step 302. During this step 302, the client device selects a simulation mode. The simulation mode includes either the simulation of the chemical operation using the algebraic-differential equation or using the explicit algebraic equation.

During step 303, the selection module 202 communicates to the processing module the selected simulation mode and the receiving module transmits 304 to the processing module the equation in accordance with the selected simulation mode.

If a simulation based on an explicit algebraic equation is selected for the first operation, the processing module performs the equation creation step 305 of creating a first algebraic-differential equation that relates a first vector of input state variables; and a first vector of internal state variables of the said first operation as an unknown of the equation. Subsequently, the processing module inserts 306 into the first algebraic-differential equation the expression of the vector of output state variables of the said first operation according to the said first explicit algebraic equation as a vector of pseudo internal state variables of the said first operation, the steady-state solution of the said algebraic-differential equation thus converging to the said first vector of output state variables according to the first explicit algebraic equation. The processing module then sets the time constant in the step 307. This time constant is set so as to be much less than the characteristic time of the first operation. The characteristic time of a chemical or biochemical operation typically varies from a few minutes to a few hours. Under these conditions, if the time constant is equal to one second, everything occurs as if the algebraic-differential system had converged instantaneously to the solution of the explicit algebraic equation. In one embodiment, the time constant is given by the user via the client module 100 and the receiving module 201 to the processing module 203 which then sets the time constant of the equation to the value indicated by the user.

The processing module then transmits the algebraic-differential equation thus obtained to the equation solving module for solving of the equation 309 in order to obtain the vector of output state variables of the first operation.

According to one embodiment, the equation solving module transmits this vector of output state variables of the first operation to the client device during a step 310.

According to one embodiment where the method comprises two operations of which a second operation is modelled from the start by an algebraic-differential equation, the processing module is configured so as to receive from the receiving module the second algebraic-differential equation during a step 304 and to merge the second algebraic-differential equation with the first algebraic-differential equation during a step 308. The processing module then transmits the system of algebraic-differential equations to the equation solving module during a step 309 which will solve this system and obtain a vector of output state variables of the process comprising the two operations.

According to one embodiment where an operation is represented by both an explicit algebraic equation and a differential algebraic equation, the client device selects a simulation mode at the step 302. If the simulation using the explicit algebraic equation is selected, the processing module performs the steps of creation 305, of insertion 306 and setting of the time constant 307 then the step of merging 308 with the algebraic-differential equations from the other operations of the process. If the simulation using the algebraic-differential equation is selected, the processing module directly merges this algebraic-differential equation with the algebraic-differential equations of the other operations of the process.

Thus, the present invention provides the means to combine a precise simulation using an algebraic differential equation for certain critical operations and the selecting of a less precise simulation using an explicit algebraic equation. The solution of this explicit algebraic equation being integrated into an algebraic-differential equation in order to allow the merging of the algebraic-differential equations into a single solving system.

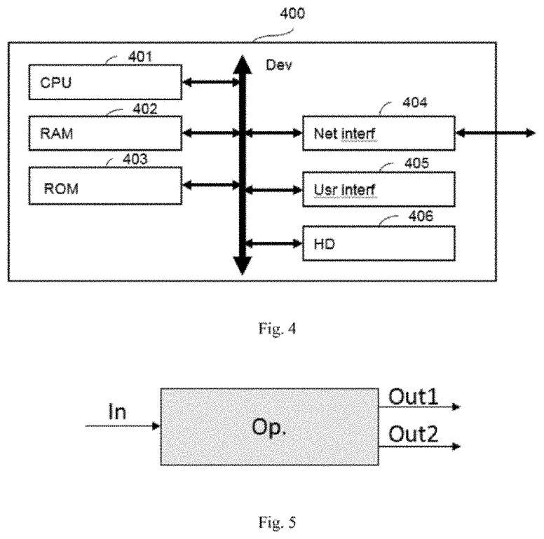

The various parts of the system described here above may be implemented by one or more computer programs. Thus, each module may correspond to a routine of one or more computer programs. The system is then implemented by a global device 400 as illustrated in FIG. 4.

The device 400 comprises a communication bus connected to:
a central processing unit 401 such as a microprocessor, otherwise referred to as CPU;
a random access memory 402, otherwise referred to as RAM, for storing an executable code of the method of the embodiments of the invention as well as the registers that are suitable for recording the variables and parameters necessary for the implementation of the method in accordance with the embodiments, the capacity of the memory storage unit may be increased by means of an optional RAM connected for example to an expansion port;
a read-only memory 403, otherwise referred to as ROM, for storing the computer programs used to implement the embodiments of the invention;
a network interface 404, which is typically connected to a communication network over which digital data to be processed is transmitted or received. The network interface 404 may be a single network interface or be composed of a set of different network interfaces (for example, wired and wireless interfaces, or different kinds of wired or wireless interfaces). The data are written to the network interface for transmission or read from the network interface for reception under the control of the software application running in the CPU 401;
a user interface 405 for receiving the inputs entered by a user or for displaying of information to the user;
a hard disk 406 otherwise referred to as HD
an input/output module 407 (otherwise referred to as I/O) for sending/receiving data to/from devices such as a video source or a display screen.

The executable code can be stored either in the read-only memory 403, or on the hard disk 406, or on a removable digital medium such as a disk for example. According to one variant, the executable code of the programs may be received by means of a communication network, via the network interface 404, in order to be stored on one of the storage media of the communication device 400, such as the hard disk 406, before being executed.

The central processing unit 401 is adapted so as to control and direct the execution of the instructions or the software code portions of the program in accordance with the embodiments of the invention, the said instructions are stored on one of the aforementioned storage media. After being powered up, the CPU 401 is capable of executing the instructions from the main RAM 402 in connection with a software application for example after these instructions have been loaded from the ROM 403 program or on the hard disk (HD) 406. This software application, when executed by the CPU 401, leads to the steps of the method being implemented in accordance with the embodiments.

### Examples

In this example the operation considered is a reaction operation (Op.) with separation of the downstream products as illustrated in FIG. 5. The reaction is as follows: S1+S3→S2+S4 with total consumption of S3. The input quantities of the reactor are given by the following:

| | Input (or Initial) |
|---|---|
| Species | Quantities (In) in Moles |
| S1 | 10 |
| S2 | 0 |
| S3 | 5 |
| S4 | 0 |

If the reaction is assumed to be total, then after the reaction, the quantities of the different species are given by:

| | Post Reaction |
|---|---|
| Species | Quantities in Moles |
| S1 | 5 |
| S2 | 5 |
| S3 | 0 |
| S4 | 5 |

Downstream of the reaction, the species are distributed between two outputs, referred to as Out 1 and Out 2, according to the distribution ratios given in the following table:

| | Distribution Ratio in the | Distribution Ratio in the |
|---|---|---|
| Species | First Output Stream (Out 1) | Second Output Stream (Out 2) |
| S1 | 10% | 90% |
| S2 | 5% | 95% |
| S3 | 50% | 50% |
| S4 | 95% | 5% |

The distribution ratios of a species give the fractions of that species contained in the different output streams. For each species, the sum of the distribution ratios equals 100%.

The vectors of input state -, first output state -, and second output state variables contain the numbers of moles of the different species and are respectively:

$${Y}_{E} = ( \begin{matrix}{m}_{S 1}^{in} \\ {m}_{S 2}^{in} \\ {m}_{S3}^{in} \\ {m}_{S4}^{in}\end{matrix} ) , {Y}_{S 1} = ( \begin{matrix}{m}_{S 1}^{out 1} \\ {m}_{S 2}^{out 1} \\ {m}_{S 3}^{out 1} \\ {m}_{S 4}^{out 1}\end{matrix} ) , {Y}_{S 2} = ( \begin{matrix}{m}_{S 1}^{out 2} \\ {m}_{S 2}^{out 2} \\ {m}_{S 3}^{out 2} \\ {m}_{S 4}^{out 2}\end{matrix} ) \vert$$

The different vectors of state variables may be determined:

At the input (initial state) of the system

$${Y}_{E} = ( \begin{matrix}10 \\ 0 \\ 5 \\ 0\end{matrix} )$$

After the reaction (before separation):

$${Y}_{Post Reaction} = ( \begin{matrix}5 \\ 5 \\ 0 \\ 5\end{matrix} )$$

At the output (final state) by applying the separation ratios to the post reaction mixture:

$${Y}_{S 1} = ( \begin{matrix}0.5 \\ 0.25 \\ 0 \\ 4.75\end{matrix} ) , {Y}_{S 2} = ( \begin{matrix}4.5 \\ 4.75 \\ 0 \\ 0.25\end{matrix} )$$

Thus the following explicit algebraic equations (Ex1, Ex2) are obtained:

$$\begin{matrix}{A}_{S 1} = ( \begin{matrix}0.1 & 0 & - 0.1 & 0 \\ 0 & 0.05 & 0.05 & 0 \\ 0 & 0 & 0 & 0 \\ 0 & 0 & 0.95 & 0.95\end{matrix} ) ; {Y}_{S 1} = {A}_{S 1} {Y}_{E} & ( Ex 1 ) \\ {A}_{S 2} = ( \begin{matrix}0.9 & 0 & - 0.9 & 0 \\ 0 & 0.95 & 0.95 & 0 \\ 0 & 0 & 0 & 0 \\ 0 & 0 & 0.05 & 0.05\end{matrix} ) ; {Y}_{S 2} = {A}_{S 2} {Y}_{E} & ( Ex 2 )\end{matrix}$$

In this example, it is considered that during the separation the species are routed before output thereof to two perfectly homogeneous cells of which the numbers of moles of the different species are grouped together in the vectors and \tilde{X}₁ and \tilde{X}₂. It is also considered that the material withdrawn from the cells through the outputs (Out 1 and Out 2) is of the same composition as that of the cells. Thus the following may then be written:

Y_{S1}=\tilde{X}₁ et Y_{S2}=\tilde{X}₂.

The system (Ex1) (Ex2) can be transformed into a differential system by introducing the pseudo-internal state variables and by setting:

$$\begin{matrix}- θ \frac{d {\tilde{X}}_{1}}{d t} - {\tilde{X}}_{1} + {A}_{S 1} {Y}_{E} = 0 and & ( Ex 3 ) \\ - θ \frac{d {\tilde{X}}_{2}}{d t} - {\tilde{X}}_{2} + {A}_{S 2} {Y}_{E} = 0 ; & ( Ex 4 )\end{matrix}$$

where θ is the arbitrary time constant of homogeneous cells; when t becomes much greater than θ, the differential term is cancelled out and the pseudo state variables converge to:

\tilde{X}₁=A_{S1}Y_{E} and \tilde{X}₂=A_{S2}Y_{E}

In order to find (Ex1) and (Ex2), it suffices to set:

Y_{S1}=\tilde{X}₁ and Y_{S2}=\tilde{X}₂.

By choosing a low time constant, it is thus possible to go from the explicit algebraic equations (Ex1 and Ex2) to the differential equations (Ex3 and Ex4). These differential equations, involving pseudo-internal state variables, can then be introduced into the system of differential equations that simulate the entire chemical or biochemical process.

1. A system for the computer-implemented simulation of a chemical or biochemical process that comprises at least a first chemical or biochemical operation; the said system comprising:
an equation solving module (resolution module) which is configured so as to solve algebraic-differential equations,
a receiving module configured so as to receive a first explicit algebraic equation that represents the said first chemical or biochemical operation, and relates a first vector of input state variables representing the initial chemical variable values of the said first operation to a first vector of output state variables representing the final chemical variable values of the said first operation; and
a processing module configured so as to:
in a first algebraic differential equation that relates:
the said first vector of input state variables; and
a first vector of internal state variables of the said first operation as an unknown of the equation;
insert into the said first algebraic-differential equation the expression of the vector of output state variables of the said first operation according to the said first explicit algebraic equation as a vector of pseudo internal state variables of the said first operation, the steady-state solution of the said algebraic-differential equation thus obtained thus converging to the said first vector of output state variables according to the first explicit algebraic equation;
set a time constant of the said algebraic-differential equation thus obtained so as to be less than a characteristic time of the said first operation; and
run the equation solving (resolution) module on the differential algebraic equation thus obtained in order to calculate the said first vector of output state variables.

2. The system according to claim 1 further comprising a selection module configured so as to select a simulation mode and in which the processing module is configured so as to perform the said insertion in accordance with a selected mode.

3. The system according to claim 1 for the computer-implemented simulation of a chemical or biochemical process that further comprises a second chemical or biochemical operation, wherein:
the said receiving module is in addition configured so as to receive a second algebraic differential equation that represents the said second chemical or biochemical operation and relates:
a second vector of input state variables representing the initial chemical variable values of the said second operation; and
a second vector of internal state variables of the said second operation as an unknown of the equation;
the steady-state solution of the said second algebraic-differential equation converging to a second vector of output state variables representing the final chemical variable values of the said second operation;
the said processing module is in addition configured so as to:
merge the said first and second algebraic-differential equations and
run the equation solving module over the combination of merged said first and second algebraic-differential equations thus obtained in order to calculate a vector of output state variables for/of the method.

4. A method for the computer-implemented simulation of a chemical or biochemical process that comprises at least a first chemical or biochemical operation; the said method comprising the following steps:
the receiving of a first explicit algebraic equation that represents the said first chemical or biochemical operation, and relates a first vector of input state variables representing the initial chemical variable values of the said first operation to a first vector of output state variables representing the final chemical variable values of the said first operation;
the creation of a first algebraic-differential equation that relates:
the said first vector of input state variables; and
a first vector of internal state variables of the said first operation as an unknown of the equation;
the insertion into the said first algebraic-differential equation of the expression of the vector of output state variables of the said first operation according to the said first explicit algebraic equation as a vector of pseudo internal state variables of the said first operation, the steady-state solution of the said algebraic-differential equation thus obtained converging to the said first vector of output state variables according to the first explicit algebraic equation,
the setting of a time constant of the said algebraic-differential equation thus obtained so as to be less than a characteristic time of the said first operation;
the solving of the algebraic-differential equation thus obtained in order to calculate the said first vector of output state variables.

5. The method according to claim 4, further comprising a simulation selection step for selecting a simulation mode, the steps of creating, inserting and setting of a time constant being performed in accordance with the selected mode.

6. The method according to claim 4 for the computer-implemented simulation of a chemical or biochemical process that further comprises a second chemical or biochemical operation, the said method in addition comprising the following steps:
the receiving of second algebraic differential equation that represents the said second chemical or biochemical operation relating:
a second vector of input state variables representing the initial chemical variable values of the said second operation; and
a second vector of internal state variables of the said second operation as an unknown of the equation;
the steady-state solution of the said second algebraic-differential equation converging to a second vector of output state variables representing the final chemical variable values of the said second operation;
the merging of the first and second algebraic-differential equations;
the solving of the combination of merged said first and second algebraic-differential equations thus obtained in order to calculate a vector of output state variables for/of the method.

7. A computer program product embodied on a non-transitory computer readable medium and comprising instructions which, upon the program being executed by a computer, lead to the computer carrying out the steps of the method according to claim 4.

8. (canceled)
